(19) United States
(12) Patent Application Publication
(10) Pub. No.: US 2014/0148706 A1
(43) Pub. Date: May 29, 2014

(54) METHOD AND DEVICE FOR DETECTING THERMAL COMFORT

(71) Applicant: Fraunhofer Gesellschaft Zur Förderung Der Angew. Forschung E.V., Munchen (DE)

(72) Inventors: Christoph Van Treeck, Kammlach (DE); Klaus Sedlbauer, Stuttgart (DE); Daniel Wölki, Otterfing (DE)

(21) Appl. No.: 14/126,028

(22) PCT Filed: Jun. 14, 2012
(86) PCT No.: PCT/EP2012/061375

§ 371 Date: Jan. 28, 2014

(30) Foreign Application Priority Data
Jun. 15, 2011 (DE) 102011077522.6

(51) Int. Cl. A61B 5/01; A61F 7/00; A61B 5/00

(52) U.S. Cl. CPC A61B 5/015; A61B 5/0075; A61B 5/7246; A61B 5/0077; A61F 7/0053; A61B 5/4836
USPC 600/474

(57) ABSTRACT

A device and corresponding method for detecting thermal comfort includes an infrared sensor to generate a thermographic image by detecting the temperature at a plurality of points to detect the surface temperature of at least one person; an image evaluation unit to receive the data of the infrared sensor to correlate the measured surface temperatures with a segmented physiological model of the person. The image evaluation unit determines a position in space and/or a gesture and/or anthropometric and/or morphological data of the at least one person, and a correlation unit receiving the data of the image evaluation unit to generate, on the basis of the position in space and/or the gesture and/or the anthropometric and/or morphological data of the at least one person, at least one variable representing thermal comfort.

### BACKGROUND

The invention relates to an apparatus for detecting thermal comfort that produces at least one measured variable representing thermal comfort. In addition, the invention relates to a method in which at least one measured variable representing thermal comfort is produced. Methods and apparatuses of the type cited at the outset can be used for need-oriented control or regulation of ventilation, heating or air conditioning installations in buildings, aircraft or vehicles or for the visual display of thermal comfort.

JP 7049141 A discloses an apparatus of the type in question. This takes account not only of the air temperature in the room that is to have its air conditioned but also of the humidity, the speed of an air flow and the infrared radiation emanating from the bounding surfaces of the room in order to determine the quantity of air to be supplied to the room and/or the temperature of said quantity of air. Furthermore, the temperature of the skin and clothing surfaces of at least one occupant of the room that is to have its air conditioned is measured contactlessly in order to ascertain therefrom the heat input by the person and the insulation value of the clothing. This allows the room climate to be attuned even better to the actual needs of the occupants.

However, this known method has the disadvantage that the thermographic image of the occupant initially allows no conclusions to be drawn as to thermal comfort. By way of example, it is thus possible for the room temperature to be lowered when the insulation value of the clothing worn by the occupant is very high, but it remains unclear whether this cooler temperature is tolerated or perceived to be pleasant by the occupant.

The invention is based on the object of detecting thermal comfort in automated fashion. In addition, an embodiment of the invention is based on the object of detecting the state of health of a person contactlessly.

### SUMMARY

The invention is based on the fundamental principle of detecting the surface temperature or infrared signature of persons digitally by means of an imaging thermographic method, augmenting these data with optional information from further sensor systems and combining them using a method for 3D gesticulation or motion detection. It is thus possible to associate the surface temperature information with the relevant surfaces of a segmented, three-dimensional physiological model of a human being. The temperature information, which is known on a segment-by-segment basis, can then be used to determine the perception of temperature or the perception of comfort. This can be accomplished by using an empirical comfort model that can optionally be extended by a model for detecting nonstatic processes.

The invention proposes detecting thermal comfort by using an image capture device with a prescribable capture region. The image capture device captures the motion of at least one person who is within the capture region. In some embodiments of the invention, the image capture device may contain at least one digital camera that maps a two-dimensional image of the capture region onto a semiconductor sensor. The semiconductor sensor can convert the two-dimensional image into an electrical signal that can be processed further as an analog or digital signal. The image capture device can operate in the visible spectral range and/or in the infrared spectral range. In some embodiments of the invention, the image capture device may comprise a plurality of cameras or camera systems so as in this way to allow three-dimensional motion detection in the capture region.

In some embodiments of the invention, the room captured by the image capture device may be an interior of a building, of a vehicle or of an aircraft. In other embodiments of the invention, the capture region may be a surface portion or a subvolume of an interior. In yet another embodiment of the invention, the image capture device can capture a portion of an open-air site in order to determine the thermal comfort of the persons on the open-air site.

The signal from the image capture device is supplied to an image evaluation device. The image evaluation device takes the images of at least one person that are captured by the image capture device in order to ascertain at least one position in the room and/or a gesticulation and/or a facial expression and/or anthropometric data for the at least one person. Provided that the image capture device captures images of at least one person continuously, the image evaluation device can also determine the time profile of a position in the room and/or the time profile of the gesticulation and/or the time profile of the facial expression of the at least one person. Provided that different persons are in the capture region of the image capture device, the image evaluation device can also determine anthropometric data, gesticulation and/or positions for a plurality of persons.

For the purposes of the present description, anthropometric data denote body dimensions, i.e. (length and length ratios) rather than specific body parts. Data that relate to the shape and the structure of the “human body” are referred to as morphological data and allow conclusions as to the sex of a person, for example. Anthropometric data, such as the size of a person, are data that can be used to estimate the body surface, for example. This can be used to parameterize and customize a mathematical calculation model.

The derivation of morphological data, such as the shape of body segments such as face, arms, legs, etc., for a person that are able to be captured by the image capture device makes it possible to infer the age and sex of a person, for example. Known formalisms are available for this purpose. Such data can then in turn be used as input parameters for the customization of a mathematical calculation model, e.g. of a thermophysiological model. According to the invention, the motion detection is initially independent of the anthropometry and the morphology.

In the image evaluation device, the surface temperature information from the image capture device can be associated with the surfaces of a segmented, three-dimensional physiological model of a human being. The segmentation reveals what body parts are captured, which allows the performance of differentiation between adjacent clothed and unclothed points of the body. This makes it possible to assess what percentage shares of the body are clothed on a segment-by-segment basis. In some embodiments of the invention, the differentiation and the parallel use of a thermophysiological model allow a heat flow ascertained by simulation to be used to calculate the clothing insulation value on a segment-by-segment basis. In some embodiments of the invention, surface temperatures of other regions, not captured in images, or the body core temperature can be calculated with a high level of precision by means of a thermophysiological simulation model that is used in parallel. The physiological model can be calibrated by the information about motion, morphology or anthropometry that is derived by means of gesticulation recognition. In addition, the physiological data allow statements regarding state of health or physiological heat balance to be derived. The influence of solar radiation, like other energy variables having a thermodynamic effect on the body (thermal history), is taken into account implicitly by virtue of the actual state of the body being described by the infrared data, sensor data and the data ascertained using the thermophysiological model.

Subsequently, the data provided by the image evaluation device are supplied to a correlation device that takes the position in the room and/or the gesticulation and/or the facial expression of the at least one person in order to produce at least one measured variable representing thermal comfort. In some embodiments of the invention, the temperature information known on the segment-by-segment basis can be used to determine the perception of temperature or perception of comfort. This can be accomplished using an empirical comfort model, for example a model based on ISO 14505.

In some embodiments, the image evaluation device can obtain information about the level of activity of a person from the gesticulation or motion recognition, i.e. the temporally and/or spatially resolved detection of the position of a person and of the relative positions of their body parts with respect to one another, which allows estimation of the metabolic rate, i.e. the energy turnover, of a person. By way of example, a sitting person is less active than a moving or standing person. However, driving a motor vehicle also results in a higher level of physical strain in comparison with a person in a position of rest, such as the front-seat passenger. Hence, the energy turnover of the driver is higher than that of the front-seat passenger. The level of activity can be used to detect thermal comfort. By way of example, a person at rest may perceive a higher humidity and/or a higher temperature to be pleasant than a physically active person.

In some embodiments of the invention, the gesticulation and the motion or the posture make it possible to infer the wellbeing of the person. By way of example, increasing fidgeting of the person allows thermal discomfort to be inferred. In yet another embodiment of the invention, the ventilation behavior of the person in the room can be detected. Thus, opening a window can indicate that there is an increased need for fresh air or that the temperature in the room should be lowered. In this case, the current thermal comfort is therefore low. The facial expression makes it possible to draw conclusions as to the current level of alertness and the level of activity. The facial expression gives away whether a person is weary or awake, for example by virtue of the raising of the eyelids. Rubbing of the eyes can indicate a state of weariness, whereas active gesticulation during discussions allows a general increase in the level of activity to be inferred. An indication of behavior-based thermal comfort is the discarding/donning or the general presence of an item of clothing, for example. This process can be detected by the image capture device using the methodology described above by determining the clothing insulation value by analyzing/simulating temperature and heat flow information. A model implemented in the correlation device for the purpose of recognizing behavior patterns in connection with thermal comfort can then deliver an appropriate output signal, which can then be used as a trigger for the entire comfort regulation mechanism. The basic principle of gesticulation recognition that is outlined here can easily be extended by further examples, which can then be implemented in the image evaluation device of one specific embodiment of the invention.

The inventive apparatus for detecting thermal comfort therefore does not necessarily or at least not exclusively detect the temperature of the skin surface of a person but rather detects the specific behavior and hence the effects of the current room climate on the wellbeing of the person. Hence, it is possible to determine thermal comfort with increased precision. A complete image of a person is obtained in terms of activity, motion, clothing insulation value, morphology, anthropometry and physiological heat balance, including skin/clothing surface temperatures and body core temperatures. All of this information can therefore be used to process information regarding the target-oriented regulatory actuation of room conditioning or air conditioning systems in order to achieve comfort-based, local regulation and actuation of air conditioning devices.

The measured variable obtained in this way and representing thermal comfort can be used for visual display of comfort, as a setpoint value in a ventilation, heating or air conditioning installation, as an input variable for a computation unit that determines a setpoint value for a ventilation, heating or air conditioning installation or as an actual value in a ventilation, heating or air conditioning installation. A ventilation, heating or air conditioning installation within the meaning of the present invention may comprise a personalized device for a user, for example seat heating or seat ventilation or at least one air blower associated with a person or with a group of persons. In other embodiments of the invention, a ventilation, heating or air conditioning installation can act unspecifically on an entire room, a room section, an entire building or a building section. In this case, the ventilation, heating or air conditioning installation may contain a supply air element, an exhaust air element, a heating body, a component cooling system or heating system or similar devices.

The measured variable representing thermal comfort may be a predicted mean vote (PMV) according to DIN EN ISO 7730 in some embodiments of the invention.

In some embodiments of the invention, the apparatus may furthermore contain at least one infrared sensor for detecting the temperature of the surface of the person at at least one point, wherein the data from the infrared sensor can be supplied to the correlation device. The surface temperature of the body of the person or his local skin temperature is set by the contact between the body and the microclimate that surrounds it. Hence, it is possible to determine the action of the microclimate that substantially influences thermal comfort. This further increases the precision of the measured variable representing thermal comfort.

In some embodiments of the invention, the empirical relationship between the local perception of temperature or comfort and the measured skin temperature can be ascertained by psychopsychic subject trials. Hence, it is possible to determine current thermal comfort with a high level of precision from the measured skin temperature and the behavior of the person.

In some embodiments of the invention, the infrared sensor may be set up to produce a thermographic image in which the temperature is detected at a plurality of points on the skin surface. In some embodiments, this can be effected by a plurality of infrared sensors that cover a different capture region. In other embodiments of the invention, it is possible to use a two-dimensional infrared sensor that, if need be with the aid of an imaging optical system, produces a thermographic image of the persons or the group of persons in the capture region. In this way, it is possible to detect the surface temperatures of different body regions. These may comprise surface regions of the body periphery and of the trunk and/or clothed and unclothed body parts. In some embodiments of the invention, it is possible to determine the insulation resistance of the clothing from the temperature differences or it is possible to infer the level of activity and/or the thermal history of the persons to be detected from the type of clothing. These measures therefore also increase the precision of the detection of thermal comfort.

In some embodiments of the invention, the image capture device may contain the infrared sensor. In some embodiments of the invention, this can be implemented by virtue of the image capture device being sensitive both in the visible spectral range and in the infrared spectral range and hence being simultaneously able to deliver input data for the input evaluation device and temperature data, derived from the infrared radiation, for the skin surface of the person to be detected to the correlation device.

In other embodiments of the invention, the image capture device may be sensitive exclusively in the infrared spectral range, for example in the spectral range between 5 μm and 50 μm, or between 10 μm and 40 μm. In this case, there is exclusively one thermographic image available that is supplied to the image evaluation device firstly in order to detect a position in the room and/or the gesticulation of the person and secondly in order to determine the temperatures at various points on the body tissue and/or the body core temperature. This embodiment of the invention can be designed and/or operated more easily and/or less expensively.

In some embodiments of the invention, the apparatus furthermore contains a computation device that can be used to determine the surface temperature of at least one surface element that is not arranged in the capture region of the image capture device and/or of the infrared sensor. This embodiment of the invention allows the detection of all factors influencing thermal comfort without the need for the room to be comprehensively equipped with infrared sensors or image capture devices. This allows the proposed apparatus to be implemented with relatively low complexity.

In some embodiments of the invention, the apparatus furthermore contains an air conditioning system that is able to alter the quantity of air supplied to the room, the air temperature, the humidity and/or the surface temperature of at least one surface portion of the surfaces bounding the room. The air conditioning system furthermore contains at least one control loop that can be used to regulate a surface temperature, a quantity of air, a humidity or an air temperature to a prescribable setpoint value. This control loop can have the at least one measured variable representing thermal comfort supplied to it as a setpoint or actual value. This allows the automatic customization of the room climate to the respective activity or the individual constitution or clothing of the user. In this way, it is possible to reduce the energy input for creating a comfortable room climate by virtue of the room climate always being tuned to individual needs.

In some embodiments, the proposed apparatus can alternatively or cumulatively also be used for visual presentation of thermal comfort, for example for research purposes, and/or for determining the body temperature of at least one person. In this case, sick persons in a collective of persons can be recognized or the state of health of occupants of a care home can be detected on a continual basis.

### BRIEF DESCRIPTION OF THE DRAWINGS

The invention will be explained in more detail below using exemplary embodiments that are shown in the appended figures, in which

FIG. 1 shows a block diagram of the inventive apparatus in a first embodiment,

FIG. 2 shows a block diagram of the inventive apparatus in a second embodiment, and

FIG. 3 shows a room equipped with the inventive apparatus.

### DESCRIPTION OF PREFERRED EMBODIMENTS

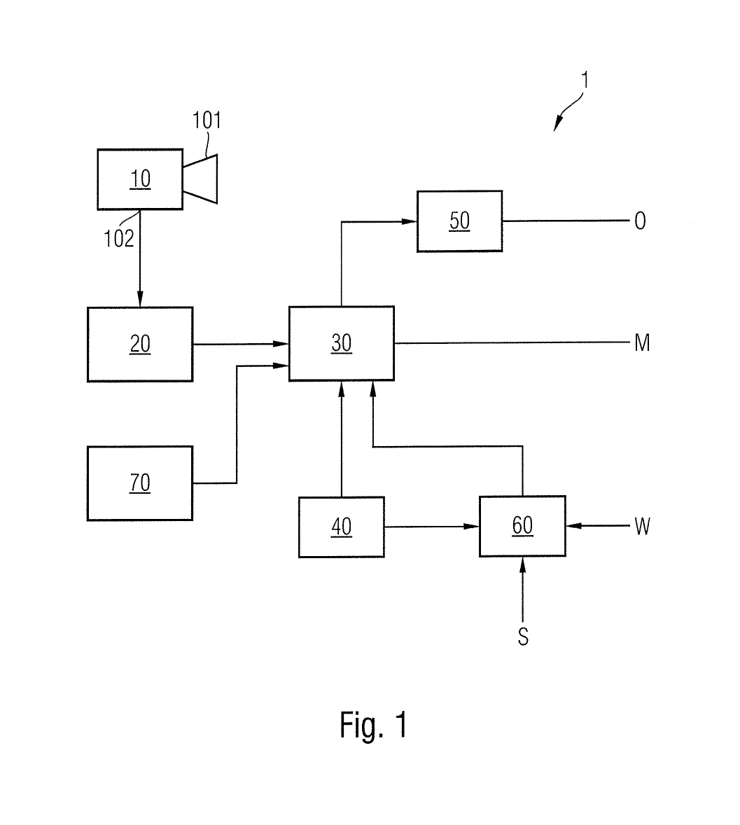

FIG. 1 shows a block diagram of a first embodiment of the present invention. The apparatus 1 contains an image capture device 10. The image capture device 10 may, in some embodiments, contain a two-dimensional image sensor, for example a CCD sensor. The region of the room that is covered by the image capture device 10 can be mapped onto the sensor by means of an imaging optical system 101.

In this case, the single image capture device 10 shown in FIG. 1 is intended to be understood only by way of example. In other embodiments of the invention, a plurality of image capture devices 10 may be present for the purpose of stereoscopic or volumetric or tomographic detection of the motion of at least one person. The single image capture device 10 shown in FIG. 1 therefore denotes a logic unit and not necessarily the number of appliances that are physically present.

The image capture device 10 is able to capture an infrared and/or visible portion of the electromagnetic spectrum. The image capture device can produce color or black-and-white or grayscale images. The image capture device 10 can produce a plurality of images cyclically or on an event controlled basis.

In some embodiments of the invention, the image capture device may contain an A/D converter, and the output 102 of said image capture device can thus provide a digital signal train representing image data.

The image data from the image capture device 10 are supplied to an image evaluation device 20. In some embodiments of the invention, the image evaluation device 20 may be in the form of a piece of software that is executed on a microprocessor or a microcontroller. In other embodiments of the invention, the image evaluation device can be implemented as an integrated circuit, for example as an ASIC and/or FPGA and/or a digital signal processor.

The image evaluation device 20 is set up to take the data stream from the image capture device in order to detect at least one position in the room and/or a gesticulation and/or facial expression and/or anthropometric data for the at least one person. Provided that the image capture device captures different images at different times, the image evaluation device can also determine the time profile of these variables. To this end, the image evaluation device can perform pattern recognition or digital image evaluation.

The data generated by the image evaluation device 20 therefore represent a motion profile for the at least one person in the room, the gesture of said person, the facial expression of said person, a posture and/or further behavior-dependent person data that are not cited here. These are subsequently supplied to the correlation device 30.

In some embodiments of the invention, the correlation device 30 may also be in the form of software that is executed on a microprocessor or microcontroller. In other embodiments of the invention, the correlation device 30 may also contain a digital signal processor, an ASIC or an FPGA. The correlation device can be implemented as a neural network or in fuzzy logic.

The task of the correlation device 30 is to take the data from the image evaluation device in order to produce at least one measured variable representing thermal comfort. By way of example, it is possible to obtain a piece of information about the level of activity of the person from the motion profile of the user in the room. In the case of a high level of activity, a lower humidity and/or a lower air temperature is generally perceived to be more pleasant. In the case of a lower level of activity, these variables can be raised in order to increase thermal comfort.

From the facial expression and/or gesticulation of the detected person, it is likewise possible to infer thermal comfort, for example in the case of increasing fidgeting, shivering or when the person takes measures to change the room climate, for example opens a window. Finally, the correlation device can infer the thermal comfort of the respective person from anthropometric data and/or morphological data and/or can deliver model parameters for a thermophysiological simulation model. In some embodiments of the invention, the anthropometric data may contain the body size of at least one person. In some embodiments of the invention, the morphological data may contain the weight of at least one person.

In addition, FIG. 1 shows an optional infrared sensor 40 that detects the temperature of the skin surface and/or clothing surface of the person. The use of an infrared sensor allows the temperature measurement to be effected contactlessly, which means that there is no need for the persons in the room to be wired with sensors. The temperature data are also supplied to the correlation device 30, which means that the latter can establish thermal comfort more quickly and/or with greater precision. The infrared sensor 40 may also contain a plurality of sensors that capture different room regions or capture identical room regions from different directions in order to allow three-dimensional temperature detection. Provided that a plurality of temperature sensors 40 are present, these can each capture a point or a room angle or determine a spatially resolved temperature distribution in their capture region.

In some embodiments, the measured values from the infrared sensor 40 can be supplied to an optional estimation device 60 that determines the clothing insulation value of the persons in the room. To this end, the estimation device 60 receives measured values from at least one clothed and one unclothed body part, which means that it is possible to infer the relevant clothing insulation value from the temperature difference and the heat flow calculated by a physiological model. The precision of the clothing insulation value can be increased if the estimation device 60 is optionally also supplied with weather data W and/or location data S. This allows a preselection to be made for the clothing insulation value and/or allows the measured value to be plausibilized. Thus, the clothing insulation value will be lower on average during warm weather than during cold weather, for example. Similarly, average users will choose different clothing in a sports hall than in a seminar room or an airport.

The estimation device 60 can also be implemented by a piece of software. The software may comprise a piece of fuzzy logic, a family of characteristic curves or a neural network. Alternatively or cumulatively, the estimation device can also be provided with hardware components, such as an A/D converter, a digital signal processor, a semiconductor memory or a microprocessor.

The precision of the clothing insulation value can be determined more precisely if the estimation device stores a thermophysiological model. Such a thermophysiological model can depict the thermoregulation properties, such as sweating, shivering, vasomotor vessel constriction and dilation, the metabolism and/or the heat balance of the human body. The presence of such a model is optional, however, and may even be dispensed with in other embodiments of the invention.

The segmentation reveals what body parts are seen using infrared, which allows the performance of differentiation between adjacent clothed and unclothed points of the body, and also an assessment to be made regarding what percentage shares of the body are clothed on a segment-by-segment basis. The differentiation and the parallel use of a thermophysiological system now also allow the clothing insulation value to be calculated on a segment-by-segment basis when the heat flow is known.

The detection of the surface temperatures at clothed and unclothed points on a person by means of the image capture device, particularly taking account of the body position, allows a mathematical model to be used to calculate heat flows that are emitted by the human body under currently prevailing ambient conditions on a specific body part and/or on the entire body. To this end, the currently measured ambient conditions (humidity, local/global surface temperatures, local/global air speeds, seat surface temperatures, etc.) are transferred to the mathematical model as input parameters.

The mathematical model itself is based on the human thermoregulation system and maps all thermoregulation mechanisms (sweating, shivering, vessel constriction/dilation) using known and validated (Foda et al., 2011) regression functions.

The nature of the active thermoregulation mechanisms (either shivering, sweating, etc.) and the intensity thereof are highly dependent on the prevailing ambient conditions, as in the case of real human beings. As a consequence of these active regulatory mechanisms, the mathematical model encounters relevant skin surface temperatures that can then be compared with the measured temperatures on clothed and unclothed surface segments of the person, since the segmenting reveals the share of the body that is clothed segment by segment or globally (in this regard see also clothing insulation value, chapter 1.1).

If the differences between measured and calculated temperatures are within a certain tolerance level, it is possible to calculate heat flows that result from the relevant temperature gradients. Comparison of the calculated heat flows from the mathematical model and of the heat flows calculated from the measurement of the surface temperatures allows precision to be improved further.

Known temperatures and heat flows can be used to calculate the clothing resistance, which can then be converted into a clothing insulation value.

Since the clothing insulation value influences thermal comfort, the measured variable M provided by the correlation device 30 can have improved precision if the correlation device 30 is provided with the output value from the estimation device 60.

Finally, some embodiments of the invention may have provision for a computation device 50 to which data from the correlation device 30 are supplied and that is set up to determine the surface temperature O of at least one surface element that is not arranged in the capture region of the image capture device and/or of the infrared sensor 40. This allows the number of sensors to be reduced without adversely affecting the precision of the method.

Finally, the correlation device 30 can also be supplied with measured values for the current room climate that are captured using sensors 70 that detect an air temperature and/or a humidity and/or a flow rate and/or a quantity of air.

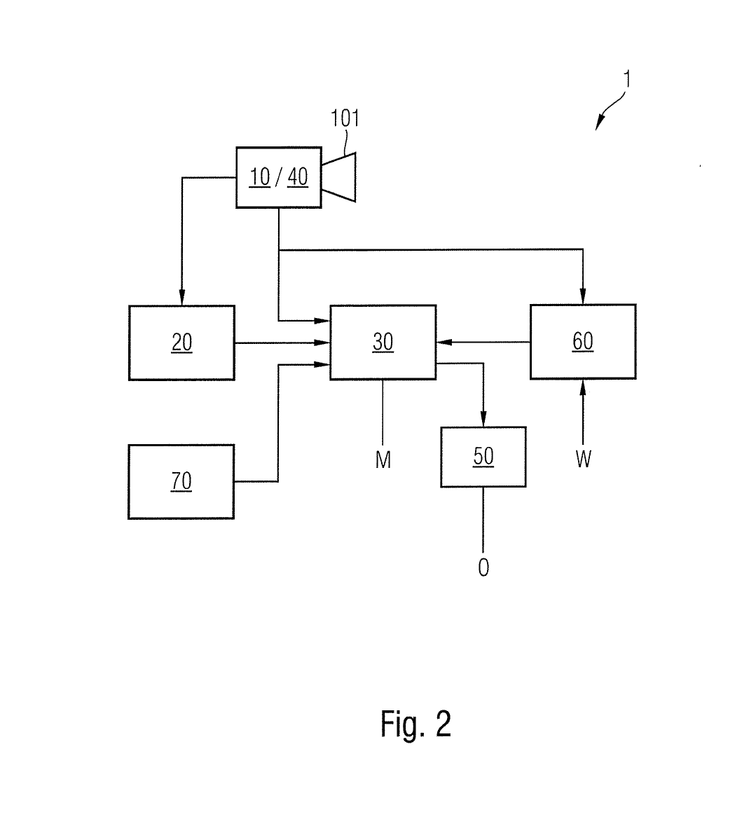

FIG. 2 shows a block diagram of a further embodiment of the invention. Identical elements are provided with the same reference symbols as in FIG. 1. Therefore, the description below is limited to the differences between the two embodiments.

As can be seen from FIG. 2, the image capture device contains an infrared sensor 40. This can be implemented in different ways. By way of example, the room region to be captured can be mapped onto a plane by at least one optical element 101. The beam path of the optical element 101 may contain a beam splitter, as a result of which at least the infrared component of the spectrum hits the infrared sensor 40 and at least the visible component of the spectrum hits the image capture device 10.

In another embodiment of the invention, the image capture device 10 and the infrared sensor 40 may be identical, as a result of which the image capture device also captures the motion of at least one person. In this case, the motion can be captured exclusively by capturing the infrared component of the electromagnetic spectrum. This makes it possible to dispense with an additional sensor for image capture.

Since the room angle captured by the image capture device 10 and the infrared sensor 40 is identical in both cases, each image element can be assigned a temperature. This allows a distinction to be drawn between clothed and unclothed body parts with greater precision using the motion pattern. At the same time, the apparatus 1 can be integrated in a room less conspicuously on account of the smaller number of appliances.

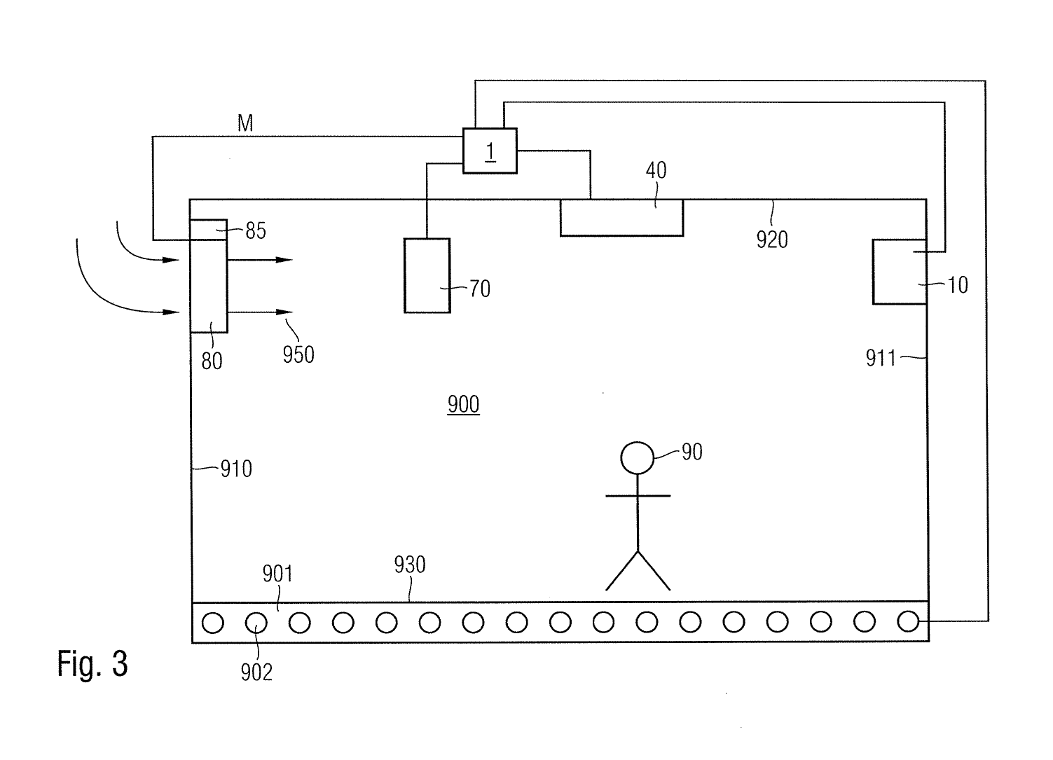

FIG. 3 shows the integration of the apparatus 1 in a room 900 by way of example. The room 900 shown in FIG. 3 is an interior in a building. The room 900 is therefore bounded by a ceiling 920, walls 910 and a floor 930. The floor 930 is covered with a screed 901 into which a heating and/or cooling device 902 has been integrated in order to influence the temperature and hence the emitted heat radiation of the floor 930. In other embodiments of the invention, a similar heating and/or cooling device may also be arranged in a wall or ceiling. In one embodiment of the invention, the heating and/or cooling device 902 may contain fluid channels that carry a heat transfer medium at relatively high or relatively low temperature in order to raise or lower the temperature. In yet another embodiment of the invention, the fluid channels 902 or the heating device may also be dispensed with. In a manner similar to that shown in FIG. 3 for the room 900 in a building, the inventive apparatus can also be operated inside a vehicle or an aircraft or a ship. In this case, it is also possible for personalized heating, ventilation or cooling devices to be controlled or regulated, such as seat heaters or air vents.

In addition, the room 900 is equipped with an optional air conditioning system 80 that can produce an air stream 950 inside the room. The quantity of the air stream 950 can be used to influence the air exchange rate and the flow rate inside the room 900. The temperature of the entering air 950 can be used to heat or cool the room 900.

The actual values of the air temperature, the flow rate, the humidity and/or the quantity of air are captured by capture means 70. The measured values are supplied to the apparatus 1, as explained above with reference to FIGS. 1 and 2.

In addition, the room 900 contains at least one image capture device 10 that can be used to capture the motion of at least one person 90. The data from the image capture device 10 are also supplied to the apparatus 1, as already explained above. Finally, the room 900 contains at least one infrared sensor 40 that covers at least a subvolume or a surface portion of the floor area of the room 900. The infrared sensor 40 can firstly detect the temperature of the surface of the person 90 at at least one point and supply this temperature to the apparatus 1. Secondly, the infrared sensor 40 can also detect the temperature of the floor 930 and/or of the walls 910. In addition, surface regions 911 may also be present, the temperature of which is not measured directly by an infrared sensor 40.

The apparatus 1 can now determine climate data for which the comfort of the person 90 is at a maximum. The climate data, i.e. the humidity, the air temperature or else the flow rate, for which the maximum comfort is obtained may be dependent on the number of persons 90, the sex thereof, the clothing thereof, the age thereof or the physical activity. In this case, the apparatus 1 can take said variables as a basis for producing different setpoint value defaults for the room climate and can output control or regulatory signals to the control of regulatory device 85 that actuate the air conditioning system 80 and/or the heating device 902, as a result of which the desired climate is obtained.

This optimizes the subjectively perceived comfort of the at least one person 90 without the need for manual intervention in the air conditioning system 80 or the heating or cooling device 902.

It goes without saying that the invention is not limited to the embodiments shown in the figures. The description above is therefore intended to be regarded not as limiting but rather as explanatory. The claims that follow are intended to be understood to mean that a cited feature is present in at least one embodiment of the invention. This does not exclude the presence of further features. Provided that the claims and the above description define “first” and “second” features, this label serves to distinguish between two features of the same type without stipulating an order of priority.

1. An apparatus for detecting thermal comfort, comprising:
an infrared sensor being adapted to produce a thermographic image by detecting the temperature at a plurality of points in order to determine the surface temperature of at least one person,
an image evaluation device being adapted to receive the data from the infrared sensor and that is adapted to correlate the measured surface temperatures to a segmented physiological model of the person, wherein the image evaluation device is adapted to determine a position in the room and/or a gesticulation and/or anthropometric and/or morphological data for the at least one person,
a correlation device being adapted to receive the data from the image evaluation device, and being adapted to provide at least one variable that represents thermal comfort from the position in the room and/or the gesticulation and/or the anthropometric and/or the morphological data for the at least one person.

2. The apparatus according to claim 1, comprising further at least one image capture device for detecting the gesticulation and/or the facial expression and/or the motion of at least one person in the room.

3. The apparatus according to claim 1, wherein the image evaluation device and/or the infrared sensor is/are adapted to determine anthropometric data and/or gesticulation and/or positions for a plurality of persons.

4. The apparatus according to claim 1, wherein the image capture device and/or the infrared sensor is/are adapted to capture images of at least one person continuously, as a result of which the image evaluation device can be used to determine the time profile of a position in the room and/or the time profile of the gesticulation and/or the time profile of the facial expression of the at least one person.

5. The apparatus according to claim 1, wherein the image capture device forms an integral part of the infrared sensor.

6. The apparatus according to claim 1, comprising further a computation device being adapted to determine the surface temperature of at least one surface element that is not arranged in the detection range of the image capture device and/or of the infrared sensor.

7. The apparatus according to claim 1, also containing comprising further an air conditioning system comprising at least one control loop to which the at least one variable representing thermal comfort can be supplied as a setpoint value or actual value.

8. The apparatus according to claim 2, comprising further an estimation device that is adapted to determine the clothing insulation value for the person, and to which at least weather data and/or location data and/or data from the infrared sensor can be supplied, wherein the data from the estimation device can be supplied to the correlation device.

9. The apparatus according to claim 8, wherein the estimation device stores a thermophysiological model.

10. The apparatus according to claim 1, wherein the image evaluation device is adapted to determine the level of activity of at least one person.

11. The apparatus according to claim 1, wherein the image evaluation device is adapted to perform morphological and/or anthropometric differentiation of the measured variable representing thermal comfort.

12. The apparatus according to claim 11, wherein the image evaluation device is adapted to perform a gender-specific determination of the variable representing thermal comfort.

13. The apparatus according to claim 1, wherein the image evaluation device is adapted to determine locally different variables representing thermal comfort.

14. The apparatus according to claim 1, wherein the image evaluation device is adapted to ascertain the thermal history of the person from level of clothing and/or external temperature and/or gender-specific estimates and/or age-specific features and/or anthropometric features and/or morphological features and/or through the parallel application of a thermophysiological model.

15. A method for detecting thermal comfort, comprising the following steps:
a thermographic image is produced in order to detect the surface temperature of at least one person by detecting the temperature at a plurality of points using an infrared sensor,
the data from the infrared sensor are supplied to an image evaluation device, a segmented surface model of a person is produced from the surface temperature, and a position in the room and/or a gesticulation and/or anthropometric and/or morphological data for the at least one person is/are determined,
the data from the image evaluation device are supplied to a correlation device, which is used to take the position in the room and/or the gesticulation and/or the anthropometric data and/or the morphological data for the at least one person in order to produce at least one variable representing thermal comfort.

16. The method according to claim 15, comprising further the following step: the gesticulation and/or the facial expression and/or the motion of at least one person in the room is/are detected by means of at least one image capture device, and the data from the image capture device are supplied to the correlation device.

17. The method according to claim 15, also comprising further the following step: a clothing insulation value for the person is determined using an estimation device to which at least weather data and/or location data and/or the data from the infrared sensor are supplied, wherein the output data from the estimation device are supplied to the correlation device.

18. The method according to claim 15, wherein the level of activity of at least one person is determined.

19. The method according to claim 16, wherein at least one temperature difference between a clothed and an unclothed body part of at least one person is determined and the temperature differences and the equivalent heat flow, calculated using a physiological model, are taken in order to determine the insulation resistance of the clothing.

20. The method according to claim 15, wherein at least one temperature difference between a clothed and an unclothed body part of at least one person is determined and the type of clothing is taken in order to infer the activity level and/or the thermal history of the at least one person.

21. The method according to claim 15, wherein a thermal comfort is visually presented and/or a body temperature of said person is determined and/or a room climate is controlled or regulated.
